(19) United States
(12) Patent Application Publication
(10) Pub. No.: US 2022/0143046 A1
(43) Pub. Date: May 12, 2022

(54) COMPOSITION AND METHODS FOR RECTAL DELIVERY OF 4-AAPA AND 5-ASA COMPOUNDS

(71) Applicant: 9 METERS BIOPHARMA, INC., RALEIGH, NC (US)

(72) Inventors: Balasingham RADHAKRISHNAN, Raleigh, NC (US); JAY P. MADAN, Raleigh, NC (US); Sireesh APPAJOSYULA, Raleigh, NC (US); Sandeep LAUMAS, Raleigh, NC (US)

(21) Appl. No.: 17/602,848

(22) PCT Filed: Apr. 13, 2020
(86) PCT No.: PCT/US20/27952

§ 371 Date: Oct. 11, 2021

Related U.S. Application Data
(60) Provisional application No. 62/832,500, filed on Apr. 11, 2019.

(51) Int. Cl. A61K 31/655; A61K 31/195; A61K 9/02; A61K 9/06; A61K 9/12

(52) U.S. Cl. CPC A61K 31/655; A61K 31/195; A61K 9/122; A61K 9/06; A61K 9/02

(57) ABSTRACT

The invention, in various aspects and embodiments, provides compositions and methods for treating or preventing inflammation of the lower gastrointestinal tract. The compositions and methods in various embodiments relate to rectal administration of pharmaceutical compositions that comprise an agent that forms one or more 4-AAPA compounds or 5-ASA compounds by azo reduction. In various embodiments, the compositions and methods treat, reduce, or prevent inflammation of the colon and/or rectum, as well as in some embodiments, an ileo-anal pouch in a subject. Compositions for rectal administration can be formulated as emulsions, suppositories (both suspension and solid), gels, and foam and foamable pharmaceutical compositions.

### CROSS-REFERENCE TO RELATED APPLICATIONS

This application claims the benefit of, and priority to, U.S. Provisional Application No. 62/832,500, filed Apr. 11, 2019, the content of which is herein incorporated by reference in its entirety.

### FIELD

In various aspects and embodiments, the invention relates to pharmaceutical compositions for the treatment and/or management of disorders involving inflammation of the lower gastrointestinal system. The invention includes treatment of conditions including but not limited to pouchitis, by rectal administration of 4-AAPA and 5-ASA azo compounds into the lower gastrointestinal tract.

### BACKGROUND

Conditions of the large intestines, such as ulcerative colitis, Crohn's disease, colon cancer, toxic megacolon and familial adenomatous polyosis (FAP) may require the surgical removal of the colon. In patients who have undergone colectomy, an ileo-anal pouch can be constructed such that an ileal reservoir is formed, allowing the patient to maintain their natural anus and avoid a permanent stoma. However, about half of these patients suffer from acute or chronic inflammation of the ileo-anal pouch. Symptoms of pouchitis can include diarrhea, increased stool frequency, urgency in passing stools, discomfort while passing stools, nocturnal seepage, incontinence, abdominal cramping, pelvic discomfort, nausea and pain. Pouchitis impairs the function of the ileo-anal pouch, decreases the quality of life of the patient and can lead to pouch failure.

Antibiotics, such as ciprofloxacin, rifaximin, tinidazole and/or metronidazole are common front line treatments for pouchitis. Patients who respond to antibiotic treatment can be placed on long-term maintenance therapy with either antibiotics or probiotics to remain in remission. Side effects from long term antibiotic use can be severe, including seizure and nerve damage. Overuse of antibiotics can shift the microflora responsible for pouchitis from conventional to nonconventional forms, such as C. difficile or fungi, resulting in pathogen induced antibiotic-resistant pouchitis. Treatment options can be pathogen specific, and can include, for example, ganciclovir, vancomycin, or fecal transplant. About 60% of patients who experience acute pouchitis will experience a relapse, and approximately 20-30% of them will develop a frequently relapsing form or antibiotic-refractory pouchitis.

Compositions and methods for treating pouchitis and other conditions of the lower gastrointestinal tract are needed, including compositions and methods that avoid or limit chronic antibiotic administration, and/or which are effective for antibiotic-refractory disease.

### SUMMARY

The invention, in various aspects and embodiments, provides compositions and methods for treating or preventing inflammation of the lower gastrointestinal tract. The compositions and methods in various embodiments relate to rectal administration of pharmaceutical compositions that comprise an agent that forms one or more 4-AAPA compounds or 5-ASA compounds by azo reduction. In various embodiments, the compositions and methods treat, reduce, or prevent inflammation of the colon and/or rectum, as well as in some embodiments, an ileo-anal pouch in a subject.

In various embodiments, the methods and compositions can reduce the incidence of pouchitis or chronic pouchitis in subjects undergoing proctocolectomy with ileal-pouch anal anastomosis (IPAA), or in some embodiments, can reduce reliance on antibiotic therapies for subjects suffering from chronic pouchitis. In some embodiments, the compositions and methods are useful for treating or ameliorating antibiotic refractory pouchitis.

In one aspect, the invention provides a method for treating or preventing inflammation of the colon (e.g., colitis) and/or rectum, or an ileo-anal pouch in a subject in need of such treatment. The method comprises rectally administering an effective amount of a pharmaceutical composition comprising an agent that forms one or more 4-AAPA compounds or 5-ASA compounds by azo reduction. 4-AAPA azo compounds include compounds that react under physiological conditions to form or release 4-AAPA and related compounds. 4-AAPA azo compounds include, for example, 4-aminophenylacetic acid azo bonded dimer [4-(4-carboxymethyl-phenylazo)-phenyl]-acetic acid and 5-(4-carboxymethyl-phenylazo)-2-hydroxybenzoic acid (APAZA). In some embodiments, the method may comprise administering a composition comprising an effective amount of an 4-AAPA azo compound and an effective amount of a 4-AAPA compound or analog or derivative thereof.

Rectal administration can be accomplished with various dosage forms, including suppositories, liquids, suspensions, solids, creams, enemas, foams or gels comprising the pharmaceutical ingredient. In various embodiments, the pharmaceutical composition provides an effective amount of the pharmaceutical agent, including for an effective contact time, at sites of inflammation of the lower gastrointestinal tract, including the colon, the rectum, or an ileo-anal pouch where present. In some embodiments, the pharmaceutical composition comprises: at least one stabilizer or surfactant, at least one thickener or foaming agent, and at least one suspending or a solubilizing agent. In some embodiments, the pharmaceutical composition is a foam or foamable composition. Foam and foamable pharmaceutical compositions, when compared to traditional suppositories, can provide the added benefit of coating a larger area of the lower gastrointestinal tract and/or ileo-anal pouch, topically spreading along the gastrointestinal tract to reach to the splenic flexure or the distal end of an ileo-anal pouch, as well as providing an increase in residence or contact time.

In some embodiments, the subject has colitis, such as ulcerative colitis, or the subject has Crohn's disease. In some embodiments, the subject has pouchitis, which can be acute or chronic pouchitis. In some embodiments, the subject's condition is refractory to other treatments. The compositions of the invention may be administered as an adjunct or alternative therapy, including for acute pouchitis, chronic pouchitis, pouchitis that is in remission, as well as pouchitis that is not responsive to other treatments. In some embodiments, the pharmaceutical composition is administered after a surgical procedure that forms the ileo-anal pouch, and prior to development of pouchitis. For example, the therapy described herein can be initiated after restorative proctocolectomy with ileal-pouch anal anastomosis.

In various embodiments, the composition is administered at least once, twice, or three times daily, and the regimen may continue for at least about five days, at least about one week, or at least about two weeks, at least about three weeks, or at least about one month. In embodiments where the composition is administered as an adjunct to antibiotic therapy, the regimen can be administered during a cycle of antibiotic treatment (which in some embodiments will last for one to four weeks), or is administered between cycles of antibiotic treatment.

Other aspects and embodiments of the invention will be apparent from the following detailed disclosure.

### DETAILED DESCRIPTION

The invention, in various aspects and embodiments, provides compositions and methods for treating or preventing inflammation of the lower gastrointestinal tract. The compositions and methods in various embodiments relate to rectal administration of pharmaceutical compositions that comprise an agent that forms one or more 4-AAPA compounds or 5-ASA compounds by azo reduction. In various embodiments, the compositions and methods treat, reduce, or prevent inflammation of the colon and/or rectum, as well as in some embodiments, an ileo-anal pouch in a subject. Compositions for rectal administration can be formulated as emulsions, suppositories (both suspension and solid), gels, and foam and foamable pharmaceutical compositions.

In various embodiments, the methods and compositions can reduce the incidence of pouchitis or chronic pouchitis in subjects undergoing proctocolectomy with ileal-pouch anal anastomosis (IPAA), or in some embodiments, can reduce reliance on antibiotic therapies for subjects suffering from chronic pouchitis. In some embodiments, the compositions and methods are useful for treating or ameliorating antibiotic refractory pouchitis.

In one aspect, the invention provides a method for treating or preventing inflammation of the colon (e.g., colitis) and/or rectum, including an ileo-anal pouch in a subject in need of such treatment. The method comprises rectally administering an effective amount of a pharmaceutical composition comprising an agent that forms one or more 4-AAPA compounds or 5-ASA compounds by azo reduction. The composition may further comprise one or more 4-AAPA compounds, which does not require azo reduction for activity.

U.S. Pat. Nos. 6,903,082, 7,425,578, and 8,048,924, which are hereby incorporated by reference in their entireties, describe methods and compositions for the treatment of inflammatory conditions of the gastrointestinal tract in which 5-ASA compounds are conjugated via an azo bond to 4-aminophenylacetic acid (4-AAPA) compounds. The azo bond can be cleaved by enzymes secreted by bacteria in the lower gastrointestinal tract, yielding pharmaceutically active 5-ASA and 4-APAA compounds. One such compound, INN-108, (also known as APAZA), is a potent inhibitor of toxin A-induced colonic inflammation in rats. See also McVey et al. Digestive Diseases and Sciences 50: 565-573 (2005). Studies indicate that 5-ASA and 4-APAA inhibit intestinal inflammation by different mechanisms and have synergistic effects when administered together in a compound such as INN-108. An oral formulation of INN-108 has successfully completed two Phase 1 clinical trials for the treatment of mild to moderate ulcerative colitis with no evidence of systemic absorption.

5-ASA is easily degraded in the stomach, such that maintaining an effective dose in the ileo-anal pouch can be difficult with oral administration. There is a pressing need for formulations that can deliver effective amounts of anti-inflammatory agents such as 5-ASA and 4-AAPA compounds, to inflamed tissue such as the colon, rectum, or an ileo-anal pouch. Such formulation should provide sufficient coverage of the inflamed tissue to achieve efficacy.

The term “lower gastrointestinal tract” as used herein includes the lower part of the small intestine, the rectum, and the large intestine (colon) and/or ileo-anal pouch, if present.

In some embodiments, the agent is a compound of the formula:

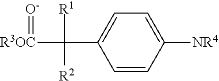

where R¹, R², and R³ are independently hydrogen, halogen, or C1 to C4 alkyl, and R⁴ is:

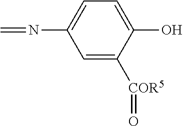

where R⁵ is selected from hydrogen, halogen, and C1 to C4 alkyl; or R⁴ is:

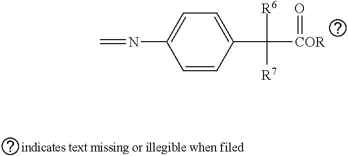

where R⁶, R⁷, and R⁸ are independently hydrogen, halogen, or C1 to C4 alkyl. For example, in some embodiments, the compound is a 4-aminophenylacetic acid azo bonded dimer. In some embodiments, the compound is 5-(4-carboxymethyl-phenylazo)-2-hydroxy-benzoic acid.

The term “4-AAPA compounds” as used herein includes 4-aminophenylacetic acid and related compounds, for example, 4-aminophenylacetic acid (4-AAPA):

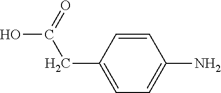 [SMILES: Nc1ccc(CC(=O)O)cc1]

as well as (4-acetylaminophenyl)-acetic acid (actarit):

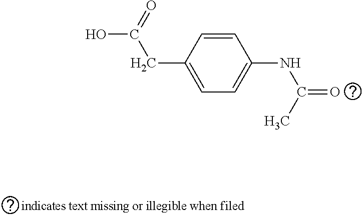

and other 4-aminophenylacetic acid derivates.

The term “5-ASA compounds” as used herein include 5-aminosalicylic acid (mesalamine) and related compounds, and compounds that react under physiological conditions to form or release 5-ASA and related compounds.

The term “4-AAPA azo compounds” as used herein includes compounds that react under physiological conditions to form or release 4-AAPA and related compounds. 4-AAPA azo compounds include, for example, 4-aminophenylacetic acid azo bonded dimer [4-(4-carboxymethyl-phenylazo)-phenyl]-acetic acid:

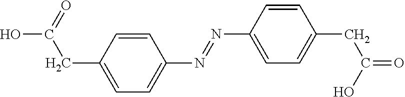 [SMILES: O=C(O)Cc1ccc(/N=N/c2ccc(CC(=O)O)cc2)cc1]

In some embodiments, the 4-AAPA compound is INN-108, 5-(4-carboxymethyl-phenylazo)-2-hydroxybenzoic acid:

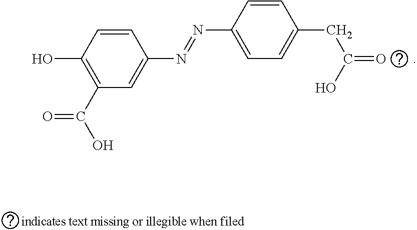 [SMILES: O=C(O)Cc1ccc(/N=N/c2ccc(O)c(C(=O)O)c2)cc1 ; "."]

The term “related compounds” as used herein includes analogs, derivatives, and compounds comprising the basic structural feature that are responsible for a compound's therapeutic activity.

For all compounds described herein, the invention includes the esters or pharmaceutically acceptable salts of the compounds.

The term “active pharmaceutical ingredient” as used herein refers to any substance or combination of substances intended to furnish pharmacological activity or to otherwise have an effect in the cure, mitigation, treatment, or prevention of disease, or to have an effect in restoring, correcting, or modifying physiological function in humans and/or animals, and is intended to cover various forms of the compound, including its esters and pharmaceutically acceptable components. An active pharmaceutical ingredient may be a combination of compounds that work in conjunction to produce a desired pharmacological effect or a prodrug that is metabolized in the body to produce one or more compounds with pharmacological effects.

The term “pharmaceutically acceptable salts” are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, liimaric acid, gluconic acid, citric acid, malic acid, ascorbic acid, benzoic acid, talmic acid, palmitic acid, alginic acid, poly-glutamic acid, naphthalenesulfonic acid, methanesulfonic acid, p-toluenesulfonic acid, naplithalenedisulfonic acid, polygalacturonic acid, and the like; and (b) salts formed from elemental anions such as chlorine, bromine, and iodine or cations such as sodium and potassium.

The term “rectal administration” means delivering a pharmaceutical composition through the anus. Rectal administration can be accomplished with various dosage forms. A suppository is a solid pharmaceutical composition. Suppositories are often small, round or cone-shaped objects. Once inserted through the anus, suppositories melt or dissolve to release active pharmaceutical ingredients. An enema is a liquid, suspension, foam or gel comprising an active pharmaceutical ingredient. In some embodiments, a liquid, suspension, or foam is delivered through the anus by a spray. Creams are semi-solid emulsions of oil and water comprising an active pharmaceutical ingredient. Creams contain more than 20% water or volatile components and typically less than 50% hydrocarbons, waxes, or polyols as vehicles. An ointment is an oil-based composition comprising an active pharmaceutical ingredient.

The term “pharmaceutically acceptable components” include salts, carriers, excipients and/or diluents of a composition or formulation according to the present invention and is a component that (i) is compatible with the other ingredients of the composition in that it can be combined with active pharmaceutical ingredients, of the present invention without rendering the active pharmaceutical ingredients unsuitable for its intended purpose, and (ii) is suitable for use with subjects as provided herein without undue adverse side effects (such as toxicity, irritation, and allergic response). Side effects are “undue” when their risk outweighs the benefit provided by the pharmaceutical composition. Non-limiting examples of pharmaceutically acceptable components include, without limitation, any of the standard pharmaceutical carriers such as phosphate buffered saline solutions, water, gels, and various types of stabilizers, foaming agents, water soluble polymers, fat bases, pH stabilizers, gelling agents, preservatives and propellants. Exemplary excipients include carbomer polymer, xanthan gum, edentate disodium, sodium metabisulfite, sodium benzoate, potassium acetate and purified water. Exemplary foamable compositions include alcohols such as ethanol and cetyl alcohol, as well as alcohol-free agents such as xanthan gum. Exemplary propellants include hydrocarbon propellant, chlorofluorocarbon propellant, hydrochlorofluorocarbon propellant, and hydrofluoroalkane propellant, as well as CO₂.

The term “effective amount” as used herein refers to an amount of a compound or composition that is sufficient to produce a desired therapeutic or preventative effect. The effective amount can vary with age, general condition of the subject, the severity of the condition being treated, the therapeutic agent administered, the formulation used, and similar factors within the knowledge and expertise of those skilled in the art. Depending on the specific condition or disease state to be treated, subjects are administered the compounds disclosed at any suitable therapeutically effective and safe dosage, which can readily be determined within the skill of the art and without undue experimentation in light of the present disclosure.

Pharmaceutical compositions that contain 4-AAPA azo compounds can facilitate the slow or delayed-release delivery of mesalamine (5-ASA) and 4-aminophenylacetic acid (4-APAA) and related compounds to the lower gastrointestinal tract with rectal administration. 4-AAPA azo compounds can be cleaved by azoreductase, an enzyme secreted by bacteria in the gastrointestinal tract. This facilitates release of the active pharmaceutical ingredients near their therapeutic target. Effective dosing can be 0.3 grams to 5 grams of a 4-AAPA azo compound per day, delivered in the form of suppositories, gels, foams, foamable emulsions and/or enemas. These formulations are an improvement over current treatments, improving their availability in the lower gastrointestinal tract and reducing adverse events associated with the systemic absorption of 5-ASA, such as interstitial nephritis which leads to irrevocable kidney damage.

In some embodiments, 4-AAPA azo compounds are suitably administered in an amount ranging from about 1 microgram per kilogram of body weight per day to about 500 milligrams per kilogram per day. In some embodiments, 4-AAPA azo compounds are suitably administered in an amount ranging from about 10 micrograms per day to about 100 milligrams per kilogram per day, or from about 100 micrograms per day to about 10 milligrams per day, or from about 500 micrograms per day to about 1 milligram per day. In some embodiments, an effective amount can be about 100 micrograms to about 100 milligrams per day, with all weights being calculated based upon the weight of 4-AAPA. The frequency of administration can be one, two, or three times per day, per week, per month, or per year or as needed to treat the condition. The duration of the treatment depends on the type of condition being treated and can be for as long as the life of the subject.

The terms “treat” or “treating” as used herein refers to any type of treatment that imparts a modulatory effect, which, for example, can be a beneficial effect to a subject afflicted with a disorder, disease or illness, including improvement in the condition of the subject (e.g. one or more symptoms), delay in the progression of the condition, and/or prevention or delay of the onset of the disorder, change in clinical parameters, disease or illness, etc.

In various embodiments, the pharmaceutical composition provides an effective amount of the active agent, including for an effective contact time, at sites of inflammation of the lower gastrointestinal tract, including the colon, the rectum, or an ileo-anal pouch where present. In some embodiments, inflammation is prominent at the inlet and outlet of the pouch. In some embodiments, the subject experiences inflammation in the reservoir of the pouch. In various embodiments, an active foaming composition can be provided for better coverage of the pouch, including inlet, outlet, or reservoir of the pouch.

In some embodiments, the pharmaceutical composition comprises: at least one stabilizer or surfactant, at least one thickener or foaming agent, and at least one suspending or a solubilizing agent. Exemplary stabilizers and surfactants can be independently selected from cetyl alcohol, coconut oil, stearic acid, hydroxypropyl methylcellulose, steric acid, citric acid, disodium ethylenediaminetetraacetic acid, and castor oil. Exemplary foaming agents and thickeners may comprise or be independently selected from poloxamer F68, polyoxyl stearyl ether, carbomer, polyethylene glycol, caprylic glycerides, cellulose, xanthan gum, and agar. For example, the foaming agent may comprise sorbitan laurate, sorbitan palmitate, laureth-4, polyglyceryl-4-isostearate, lecithin, polyoxyethylene sorbitan monostearate (TWEEN 60), polyoxyethylene sorbitan monooleate (TWEEN 80), Myri⁴⁵, Myri⁹, Myri⁵⁹, Brij38, Brij52, Brij56, sorbitan monolaurate, isoceteth-20, sodium lauryl sulfate, triethanolamine lauryl sulfate, and others. Exemplary suspending or solubilizing agents may be independently selected from propylene glycol, glycerine, emulsifying wax, and a solvent. In various embodiments, the solvent is an aqueous solvent (e.g., water). In some embodiments, the solvent comprises a lower alcohol, which may be methanol, ethanol, propanol, or isopropanol. In some embodiments, the solvent is an aqueous solvent (e.g., water) and an alcohol (e.g., lower alcohol) mixture. In some embodiments, the at least one stabilizer or surfactant is less than about 1 wt %, such as from about 0.01% to about 0.1%, or in some embodiments, about 0.05% by weight of the composition. In some embodiments, the at least one thickener or foaming agent is about 0.05% to about 4% by weight of the composition, and at least one suspending or solubilizing agent is about 0% to 30% by weight of the composition (e.g., about 10% to about 30%, or about 15% to about 30%).

In some embodiments, the pharmaceutical composition is a foam or foamable composition. Foam and foamable pharmaceutical compositions, when compared to traditional suppositories, can provide the added benefit of coating a larger area of the lower gastrointestinal tract and/or ileo-anal pouch, topically spreading along the gastrointestinal tract to reach to the splenic flexure or the distal end of an ileo-anal pouch, as well as providing an increase in residence or contact time, thereby enhancing the treatment effect of the active pharmaceutical ingredient. In some embodiments, rectal foam pharmaceutical compositions can include the administration of about 20 to about 60 mL of therapy per dose, a distinct advantage over enemas with respect to convenience and patient comfort and urgency during administration and treatment.

In some embodiments, the composition is a foamable composition that comprises a propellant. Exemplary propellants include P-22, P-142b, and P-152a, which have good miscibility with water and are therefore useful as solvents in accordance with the invention. Additional suitable propellants include P-11, P-12, and P-114, which are chlorofluorocarbon propellants with relatively low toxicity and inflammability. Particularly for aqueous compositions, P-12 (Dichlorodifluoromethane) or a mixture of P-12 and P-114 (Dichlorotetrafluoroethane) can be employed. In various embodiments, the propellant is a hydrochlorofluorocarbon (HCFC) or hydrofluorocarbon (HFC), which differ from CFCs in that they do not contain chlorine and have one or more hydrogen atoms. These compounds break down in the atmosphere at a faster rate than the CFCs resulting in a lower ozone depleting effect. In some embodiments, the composition comprises a mixture of propellants, such as a mixture of P-11 or P-14 to prepare a slurry with the active agent and/or dissolve other ingredients including surfactants, and one or more other HCFC, HFC, or HC propellants.

TABLE 1
Properties of Chlorofluorocarbon Propellants

| | | | V.P. | B.P. ° F. | Liquid Density |
|---|---|---|---|---|---|
| Name | Formula | No. | @70° F. (psia)^{a} | (1 atm) | @70° F. (g/mL) |
| Trichloromonofluoromethane | CCl₃F | 11 | 13.4 | 74.7 | 1.485 |
| Dichlorodifluoromethane | CCl₂F₂ | 12 | 84.9 | −21.6 | 1.325 |
| Dichlorotetrafluoroethane | CClF₂ClF₂ | 114 | 27.6 | 39.4 | 1.468 |

^{a}psia (pounds per square inch absolute) = psig (pounds per square inch gauge + 14.7)

TABLE 2
Properties of Hydrochlorofluorocarbon and Hydrofluorocarbon Propellants

| | | | V.P. | B.P. ° F. | Liquid Density |
|---|---|---|---|---|---|
| Name | Formula | No. | @70° F. (psia) | (1 atm) | @70° F. (g/mL) |
| Chlorodifluoromethane | CHClF₂ | 22 | −135.7 | −41.4 | 1.21 |
| Trifluoromonofluoroethane | CF₃CH₂F | 134a | 85.8 | −15.0 | 1.21 |
| Chlorodifluoroethane | CH₃CClF₂ | 142b | 43.8 | 14.4 | 1.12 |
| Difluoroethane | CH₃CHF₂ | 152a | 76.4 | −12.5 | 0.91 |
| Heptafluoropropane | CF₃CHFCF₃ | 227 | 57.7 | 2.3 | 1.41 |

In some embodiments, the composition is an aqueous composition, and comprises a hydrocarbon propellant, which have low toxicity and nonreactivity. For example, the hydrocarbons will remain on top of the aqueous layer and provide the force to push the contents out of the container. Exemplary hydrocarbon propellants include propane, butane, and isobutene. These hydrocarbon propellants are used alone or as mixtures or mixed with other liquefied gases to obtain the desired vapor pressure, density, and degree of flammability. The flammability hazard can be substantially reduced by using mixtures of propellants and with the development of newer types of dispensing valves (i.e., valve with vapor tap).

TABLE 3
Properties of Hydrocarbon Propellants

| | | | V.P. | B.P. ° F. | Liquid Density |
|---|---|---|---|---|---|
| Name | Formula | No. | @70° F. (psia) | (1 atm) | @68° F. (g/mL) |
| Propane | C₃H₈ | A-108 | 124.7 | −43.7 | 0.50 |
| Isobutane | C₄H₁₀ | A-31 | 45.1 | 10.9 | 0.56 |
| Butane | C₄H₁₀ | A-17 | 31.2 | 31.1 | 0.58 |

Pharmaceutical foam compositions generally comprise hydrophilic and/or hydrophobic solvents or liquids, emollients, co-solvents, foaming agents, gelling agents, foam adjuvants and water. Water is the primary hydrophilic solvent in most foam systems. Examples of hydrophilic solvents include glycerin, propylene glycol, hexylene glycol, diethylene glycol, terpenes, di-terpenes, limonene, terpeneol, 1-menthol, dioxolane, ethylene glycol, dimethylsulfoxide (DMSO), dimethylformamide, dimethylacetamide, azone, myristyl alcohol, lauryl alcohol, lauric acid, caprylic acid and polyethylene glycols. Hydrophobic solvents are materials with a solubility in purified water at room temperature of less than about 1 g per 1000 mL or less and are liquid at room temperature. Examples include mineral oil, triglyceride oil, olive oil, corn oil, soybean oil, canola oil, cottonseed oil, coconut oil, sesame oil, sunflower oil, borage seed oil, cod liver oil, salmon oil, flaxseed oil, wheat germ oil, evening primrose oils, omega-3 and omega-6 fatty acids, linoleic acid, linolenic acid, gamma-linoleic acid, eicosapentaenoic acid, docosahexaenoic acid and some essential oils. The creation of a foamable composition with low water content usually requires high concentrations of a foaming surfactant system, which may comprise a high proportion of ionic surfactants.

In some embodiments, the pharmaceutical composition is a gel or emulsion. The term “emulsion” means a blended composition of non-miscible components. The non-miscible components can include a lipophilic component, an aqueous component, and/or a gas component. An emulsion can be composed of any combination of these components, such as a lipophilic-in aqueous emulsion, an aqueous-in-lipophilic emulsion, or air-in lipophilic and aqueous emulsion. An emulsion in which one of the components is a foaming agent or gas can be a foam or foamable pharmaceutical composition. Preferred excipients include surfactants, especially non-ionic surfactants; emulsifying agents, especially emulsifying waxes; and liquid non-volatile non-aqueous materials, particularly glycols such as propylene glycol. The oil phase of an emulsion may contain other oily pharmaceutically approved excipients.

In some embodiments, the pharmaceutical composition is a suppository. The suppository is generally a solid dosage form. The term “solid dosage form” means blended compositions of solids in a base. A solid dosage form can be composed of non-lipophilic components and lipophilic components such as fat. Hard Fat, NF, is an exemplary component.

In some embodiments, the pharmaceutical composition is a rectal suspension. The term “suspension” means a homogeneous suspension of components in an aqueous media. For example, a suspension includes a combination of components such as aqueous soluble components, polymers, gums and preservatives.

Described herein are methods of treatment, delivery compositions, and pharmaceutical compositions for the prophylaxis or treatment of inflammatory diseases such as pouchitis, in subjects in need of such treatment. Subjects to be treated by the methods and delivery systems described include both human and non-human animals (e.g. dog, cat, cow, horse), and are preferably mammalian, and most preferably human.

In some embodiments, methods for treating pouchitis can include rectally administering an effective amount of 4-AAPA azo compounds. 4-AAPA azo compounds are metabolized by gastrointestinal microbiota by azo reduction to 5-ASA compounds and 4-AAPA compounds, such that inflammation is reduced by a topical medication action. Rectal administration can include pharmaceutical compositions that are gels, foams, liquid enemas and/or suppositories. An effective amount of 4-AAPA compounds can be an amount sufficient to deliver at least 100 milligram of 4-AAPA compounds per day, preferably 1 to 5 grams per day. 4-AAPA azo compound metabolites, such as 4AAPA compounds and 5-ASA compounds can act as innate and adaptive immune modulators, inhibiting the secretion of cytokines such as tumor necrosis factor (TNF-α), interleukin 1 beta (IL-10), interleukin-6 (IL-6), and interleukin-2 (IL-2). Thus, the pharmacological effect of immunocytokines such as interleukin-15 (IL-15) on the adaptive immune response can be reduced, thereby reducing inflammation. The administration of 4-AAPA azo compounds can include the optional co-administration of a probiotic or microbiota. 4-AAPA azo compounds may also be co-administered with (or as an alternative to) other known pouchitis treatments, such as ciprofloxacin, rifaximin, tinidazole and/or metronidazole.

In some embodiments, unit doses of the composition comprise from 0.3 mg to about 5 g of the 4-AAPA azo compound. In some embodiments, a unit dose comprises from about 0.5 to about 3 g of the 4-AAPA azo compound, or from about 0.5 to about 2 g, or from about 0.5 to about 1 g. In some embodiments, the composition comprises from about 5% to about 25% (wt %) active ingredient (e.g., about 5% to about 15%, or about 10% active ingredient), such as 4-AAPA azo compounds and optionally 4-AAPA compounds. Various dosing formulations are further described below.

In some embodiments, pharmaceutically acceptable components and concentrations can be selected to optimize delivery of active pharmaceutical ingredients by, for example, optimizing the stability, spreadability, residence time and/or contact time of active pharmaceutical ingredients in the lower gastrointestinal tract. To this end, some embodiments of the pharmaceutical composition include stabilizers and surfactants in an amount from about 0.05 to 1% by weight, per stabilizer and surfactant. In some embodiments, the pharmaceutical composition can include foaming agents and thickening agents in an amount from about 0.05% to 4% by weight, per foaming agent and thickening agent. In some embodiments, solvents can include an alcohol water mixture wherein the ratio of alcohol to water can be between approximately 1:99 and 20:80. In some embodiments, solvents can be free of alcohol. In some embodiments, the foaming agent can be free of alcohol. In some embodiments, the pharmaceutical composition can include suspending or solubilizing agent such as polymers in amount from about 0 to 30% by weight.

In some embodiments, pharmaceutical compositions can include 0.3 grams to 5 grams of 4-AAPA azo compounds as the active pharmaceutical ingredient and about 20 to about 60 milliliter of pharmaceutically acceptable components to form an emulsion, gel emulsion, foam or foamable emulsion, or liquid enema. Pharmaceutically acceptable components can include, but are not limited to, stabilizers and surfactants such as cetyl alcohol, coconut oil, stearic acid, hydroxypropyl methylcellulose (HPMC), steric acid, citric acid, disodium ethylenediaminetetraacetic acid, and/or castor oil foaming and thickening agents such as poloxamer F68, hard fat, carbomer 934P, carboplos, polyoxyl stearyl ether, polyethylene glycol (PEG), caprylic glycerides, cetyl alcohol, cellulose, xanthan gum, gelling agents and/or agar; suspending or solubilizing agents, such as water soluble polymers including propylene glycol, glycerine, emulsifying wax; and solvents such as water and alcohol. Other foaming agents that may be included comprise sorbitan laurate, sorbitan palmitate, laureth-4, polyglyceryl-4-isostearate, lecithin, polyoxyethylene sorbitan monostearate (TWEEN 60), polyoxyethylene sorbitan monooleate (TWEEN 80), Myri45, Myri49, Myri59, Brij38, Brij52, Brij56, sorbitan monolaurate, isoceteth-20, sodium lauryl sulfate, and triethanolamine lauryl sulfate. In some embodiments, the pharmaceutical composition can include the 4-AAPA azo compound INN-108. In some embodiments, the pharmaceutical composition comprises the 4-AAPA azo compound INN-108, and 4-APAA compound, which is optionally 4-Acetyl APAA (Actarit).

In an exemplary embodiment, the pharmaceutical composition comprises from 400 to 600 mg (e.g., about 500 mg) of a 4-AAPA azo compound in a mixture of ethanol and water. The composition further comprises at least 1% cetyl alcohol (e.g., from 1% to about 10%, or from 1% to 5% cetyl alcohol), disodium EDTA (e.g., up to about 1% EDTA), and propylene glycol (e.g., from 10% to 30% propylene glycol), rendering a foam or foamable composition. All amounts refer to wt %.

In another exemplary embodiment, the pharmaceutical composition comprises from 400 to 600 mg (e.g., about 500 mg) of INN-108 diacid, about 30 mL of water and 50 mL of ethanol. The composition further comprises at least 1% cetyl alcohol (e.g., from 1% to about 10%, or from 1% to 5% cetyl alcohol), disodium EDTA (e.g., up to about 1% EDTA), and propylene glycol (e.g., from 10% to 30% propylene glycol), rendering a foam or foamable composition. All amounts refer to wt %.

In another exemplary embodiment, the pharmaceutical composition comprises from about 500 mg to about 5 g of INN-108 disodium salt dissolved in water. The composition may further comprises at least 1% cetyl alcohol (e.g., from 1% to about 10%, or from 1% to 5% cetyl alcohol), disodium EDTA (e.g., up to about 1% EDTA), methylparaben, propylparaben, xanthan gum and propylene glycol, rendering a foam or foamable composition.

In some embodiments, the pharmaceutical composition is filled into an aluminum canister with an aerosol propellant. The propellants can include, for example, n-butane, isobutane and/or propane. In some embodiments, a propellant mixture of propane and isobutene can be combined at a molar ratio of about 1% to about 25% propane and from about 26% to about 99% isobutene. In some embodiments, a propellant mixture of propane, n-butane, and isobutene can be combined in a molar ratio of about 1% to about 20% propane, about 1% to about 20% n-butane, and about 10% to about 98% isobutene, respectively.

In some embodiments, suppositories can include the active pharmaceutical agent, for example 0.3 grams to 5 grams of 4-AAPA azo compounds in a solid, gel or emulsion or suspension comprised of pharmaceutically acceptable components, and can be encapsulated in a suppository base, for example, water soluble bases or hard fats having an ascending melting point of 32° C. to 35.5° C. The pharmaceutically acceptable components can include, for example stabilizers and surfactants in an amount from about 0.05 to about 1% by weight, per stabilizer and surfactant. In some embodiments, the pharmaceutical composition can include thickening agents or foaming agents, in an amount from about 0.05% to about 4% by weight, per thickening agent or foaming agent. A hard fat can include, but are not limited to, esterified mixtures of mono-, di-, and triglycerides of which are obtained by esterification of fatty acids. Other suitable suppository bases can include, but are not limited to cocoa butter, lauric oil, beef tallow, hard fat, and PEG, TWEEN, or any combination of these pharmaceutically acceptable components. The weight ratio of active pharmaceutical ingredient to hard fat can be in the range of 1:2 to about 1:1.2, and can optionally include microbiota.

In some embodiments, the pharmaceutical composition can include the active pharmaceutical ingredient, for example 0.3 grams to 5 grams of 4-AAPA azo compound, with a liquid carrier medium containing pharmaceutically acceptable components in a volume of about 60 to about 100 mL. A liquid carrier medium can include stabilizers and surfactants in an amount from about 0.05 to about 1% by weight, per stabilizer and surfactant. In some embodiments, the pharmaceutical composition can include foaming agents and thickening agents in an amount from about 0.05% to about 4% by weight, per foaming agent and thickening agent. In some embodiments, the pharmaceutical composition can include water-soluble polymers in amount from about 0 to about 30% by weight, per water soluble polymer. In some embodiments, solvents can include an alcohol water mixture wherein the ratio of alcohol to water can be between approximately 1:99 and 20:80. In some embodiments, the solvent can be a non-alcohol water mixture.

In some embodiments, 4-AAPA azo compounds are used as a delivery system to simultaneously deliver 5-ASA compounds and 4-AAPA compounds to the lower gastrointestinal tract via in vivo azo reduction of 4-AAPA azo compounds to treat inflammatory conditions of the lower gastrointestinal track, for example, an ileo-anal pouch. The administration of 4-AAPA azo compounds can include the optional co-administration of microbiota. The administration of an effective amount of 4-AAPA azo compounds to deliver at least 100 milligram per day of 4-AAPA compounds and 5-ASA compounds into the lower gastrointestinal tract can modulate innate and adaptive immune responses and reduce microbiota load, thereby reducing gastrointestinal inflammation through topical medication action by contact with the inflamed tissue.

In some embodiments, the delivery system can include the active pharmaceutical ingredient, for example 0.3 grams to 5 grams of 4-AAPA azo compounds in a gel or emulsion pharmaceutical composition, wherein the pharmaceutically acceptable components can include, for example stabilizers and surfactants, thickening or foaming agents, suspending or solubilizing agents, and can optionally include probiotics or microbiota.

For example, in a preferred embodiment, the pharmaceutical composition can include 500 mg to 5000 mg of 4-AAPA azo compounds in about 300 mL of water and 50 mL of ethanol, along with at least about 1% cetyl alcohol, up to about 1% disodium EDTA, and up to 30% propylene glycol, rendering an emulsion. With the addition of an appropriate amount of gelling agent, such as hydroxypropyl methylcellulose, a pharmaceutical gel can be formed.

In some embodiments, the delivery system can include a pharmaceutical composition, comprised of, for example 0.3 grams to 5 grams of 4-AAPA azo compound, and pharmaceutically acceptable components in a volume of about 20 to about 60 milliliters, forming an emulsion, gel emulsion, suspension, foam of formable composition. In some embodiments, the delivery system can include a pharmaceutical composition comprised of, for example about 0.3 grams to about 5 grams of 4-AAPA azo compound and pharmaceutically acceptable components in a volume of about 300 milliliters. Pharmaceutically acceptable components include, but are not limited to, stabilizers and surfactants such as cetyl alcohol, coconut oil, stearic acid, steric acid, citric acid, disodium ethylenediaminetetraacetic acid, and/or castor oil; foaming agents and thickening agents such as poloxamer F68, hydroxypropyl methylcellulose, Carobomer934P, polyoxyl stearyl ether, polyethylene glycol (PEG), caprylic glycerides, cellulose, xanthan gum, and/or agar; suspending or solubilizing agents, such as propylene glycol, glycerine and other water soluble polymers and emulsifying wax; preservatives such as methylparaben; and solvents such as water and/or alcohol.

In an exemplary embodiment, the pharmaceutical composition comprise about 500 mg of 4-AAPA azo compounds in about 10 mL of water and 1 mL of ethanol, along with at least about 1% cetyl alcohol (e.g., from about 1% to about 5% cetyl alcohol), up to about 1% disodium EDTA, and about 30% propylene glycol, rendering a foam or foamable composition.

In an exemplary embodiment, the pharmaceutical composition can comprise about 500 mg to about 5000 mg of 4-AAPA azo compounds in about 300 mL of water and about 50 mL of ethanol, along with at least about 1% cetyl alcohol (e.g., from about 1% to about 5% cetyl alcohol), up to about 1% disodium EDTA, and about 30% propylene glycol, rendering an emulsion. With the addition of an appropriate amount of gelling agent, such as gelatin, a pharmaceutical gel can be formed.

In an exemplary embodiment, the pharmaceutical composition comprises about 500 mg to about 5000 mg of 4-AAPA azo compounds in an alcohol-free aerosol foam comprising a hydrofluoroalkane propellant, an oil phase and an aqueous phase. The 4-AAPA compound may be INN-108 disodium salt, and may be dispersed in either or both phases of the emulsion. The composition further comprises one or more surfactants, emulsifiers, emulsion stabilizers, buffers, and/or other excipients as described in US 2007/0154402, which is hereby incorporated by reference in its entirety.

In some embodiments, a pharmaceutical composition for the rectal administration of 4-AAPA azo compounds can be comprised of an effective amount of 4-AAPA azo compounds, at least one stabilizer or surfactant, at least one thickening or foaming agent, and at least one suspending or solubilizing agent. For example, the pharmaceutical composition can be an emulsion that is a foam, or a foamable composition that becomes a foam when expelled under the appropriate conditions. Preferably, the pharmaceutical composition can include the 4-AAPA azo compound INN-108.

In some embodiments, a pharmaceutical composition for the rectal administration of compounds can be comprised of an effective amount of 4-AAPA azo compounds and a derivative of 4-APAA compound, at least one stabilizer or surfactant, at least one thickening or foaming agent, and at least one suspending or solubilizing agent. For example, the pharmaceutical composition can be an emulsion that is a foam, or a foamable composition that becomes a foam when expelled under the appropriate conditions. Preferably, the pharmaceutical composition can include the 4-AAPA azo compound INN-108 and 4-APAA.

In some embodiments, a pharmaceutical composition for the rectal administration of compounds can be comprised of an effective amount of 4-AAPA azo compounds and a derivative of 4-APAA compound, at least one stabilizer or surfactant, at least one thickening or foaming agent, and at least one suspending or solubilizing agent. For example, the pharmaceutical composition can be an emulsion that is a foam, or a foamable composition that becomes a foam when expelled under the appropriate conditions. In some embodiments, the pharmaceutical composition can include the 4-AAPA azo compound INN-108 and 4-Aectyl APAA (Actarit).

In some embodiments, the composition includes a 4-APAA compound or derivative thereof that does not need azo reduction for activity, along with a 4-AAPA azo compound. In these embodiments, the composition provides a bolus of active agent for immediate reduction of symptoms, along with an amount of 4-AAPA which will become active over time. In these embodiments, the composition provides a fast acting and sustainable action.

In some embodiments, the composition includes a 5-ASA compound or derivative thereof that does not need azo reduction for activity, along with a 4-AAPA azo compound. In these embodiments, the composition provides a bolus of active agent for immediate reduction of symptoms, along with an amount of 4-AAPA which will become active over time. In these embodiments, the composition provides a fast acting and sustainable action.

In some embodiments, the subject has colitis, such as ulcerative colitis, or the subject has Crohn's disease. In some embodiments, the subject has pouchitis, which can be acute or chronic pouchitis. In some embodiments, the subject's condition is refractory to other treatments.

In some embodiments, the subject has an immune-mediated form of pouchitis. For example, PSC-associated and IgG4-associated pouchitis are immune-mediated forms of antibiotic refractory pouchitis. Treatments for immune-mediated pouchitis can include steroids, immunomodulators, and anti-TNF biologics. The compositions of the invention may be administered as an adjunct or alternative therapy in these conditions.

In various embodiments, the subject is suffering from acute pouchitis. In some embodiments, this treatment may reduce active inflammation of the ileo-anal pouch, and thereby reduce symptoms associated with pouchitis, such as diarrhea, increased stool frequency, urgency in passing stools, discomfort while passing stools, nocturnal seepage, incontinence, abdominal cramping, pelvic discomfort, nausea and pain. In some embodiments, administration of the pharmaceutical composition prevents or reduces the likelihood of pouch failure.

In various embodiments, the subject is suffering from chronic pouchitis. In a subject with chronic pouchitis, the composition can be administered in a regimen to manage the condition, including during times of active inflammation as well as periods of remission. In some embodiments, administration of the pharmaceutical composition prevents or reduces the incidence of pouch failure. In some embodiments, the composition is administered during a cycle of antibiotic treatment, or between cycles of antibiotic treatment. In some embodiments, the composition is administered as an alternative to antibiotic therapy, or reduces reliance on antibiotic treatment. In some embodiments, the composition is administered with probiotic therapy.

In various embodiments, the subject has pouchitis that is in remission. In such embodiments, the composition may extend the period of remission, and prevent or reduce periods of relapse. In some embodiments, the composition is administered with probiotic therapy, as described further below.

In some embodiments, a pharmaceutical composition comprising 4-AAPA azo and/or 5-ASA azo compounds, and optionally 4-AAPA and/or 5-ASA compounds, is administered to a subject whose pouchitis is not responsive to other treatments commonly administered for the treatment of pouchitis, such as antibiotics, antivirals, corticosteroids, probiotics, non-steroidal anti-inflammatory drugs, or anti-TNF biologicals. In various embodiments, the subject is suffering from antibiotic refractory pouchitis, and the composition is administered as an alternative to antibiotic therapy.

In some embodiments, a pharmaceutical composition comprising 4-AAPA azo and 5-ASA compounds, and optionally 4-AAPA compounds, is administered to a subject suffering from pouchitis and an alternative pouchitis treatment is not administered to that subject because the alternative treatment causes an undesirable effect in that subject, such as an allergic reaction.

In some embodiments, a subject suffering from pouchitis is treated with a pharmaceutical composition comprising 4-AAPA azo and 5-ASA compounds, and optionally 4-AAPA or 5-ASA compounds, in combination with another treatment commonly administered to subjects suffering from pouchitis, such as an antibiotic, an antiviral, a corticosteroid, a probiotic, a fecal transplant, a non-steroidal anti-inflammatory drug or an anti-TNF biological. Such combinatorial treatment reduces inflammation of the ileo-anal pouch. Combinatorial treatment may provide a greater reduction in inflammation than any single treatment alone. Combinatorial treatment may ameliorate more pouchitis symptoms that treatment with any single agent alone. Combinatorial treatment may provide synergistic effects.

In some embodiments, the pharmaceutical composition is administered after a surgical procedure that forms the ileo-anal pouch, and prior to development of pouchitis. For example, the therapy described herein can be initiated after restorative proctocolectomy with ileal-pouch anal anastomosis, the surgical treatment that forms an ileo-anal pouch. Post-surgical administration may reduce residual inflammation of the ileo-anal pouch and/or may inhibit or prevent the expansion or spread of inflammation to the ileo-anal pouch. Post-surgical administration may further ameliorate the symptoms of ulcerative colitis, familial adenomatous polyposis, or Crohn's disease. Post-surgical administration of the pharmaceutical composition comprising 4-AAPA azo and 5-ASA compounds, and optionally 4-AAPA compounds, will reduce the amount of time required to recover from surgery in some embodiments, and can decrease the likelihood of pouch failure or development of chronic pouchitis. In some embodiments, the subject further recieves probiotic therapy.

Where probiotics are desired, probiotics can be selected from Saccharomyces boulardii; Lactobacillus rhamnosus GG; Lactobacillus plantarum 299v; Clostridium butyricum M588; Clostridium difficile VP20621 (non-toxigenic C. dfficile strain); combination of Lactobacillus casei, Lactobacillus acidophilus (Bio-K+CL1285); combination of Lactobacillus casei, Lactobacillus bulgaricus, Streptococcus thermophilus (ACTIMEL); combination of Lactobacillus acidophilus, Bifdobacterium bifidum (FLORAJEN3); combination of Lactobacillus acidophilus, Lactobacillus bulgaricus delbrueckii subsp. bulgaricus, Lactobacillus bulgaricus casei, Lactobacillus bulgaricus plantarum, Bifdobacterium longum, Bifdobacterium infantis, Bifdobacterium breve, and Streptococcus salivarius subsp. thermophilus (VSL #3)).

In various embodiments, the composition is administered at least once, at least twice, or at least three times daily. In some embodiments, the composition is administered for at least one week (e.g., at least once, twice, or three times daily for at least one week). In some embodiments, the regimen comprises administration for at least about two weeks, at least about three weeks, or at least about one month. In embodiments where the composition is administered as an adjunct to antibiotic therapy, the regimen can be administered during a cycle of antibiotic treatment (which in some embodiments will last for one to four weeks), or is administered between cycles of antibiotic treatment.

Thus, the invention can be embodied in many different forms, not all of which are described above, and all such forms are contemplated to be within the scope of the invention. It is emphasized that the terms “comprises” and “comprising”, when used in this application, specify the presence of stated features, steps, or components and do not preclude the presence or addition of one or more other features, steps, components, or groups thereof. The singular forms “a,” “an,” and “the” are intended to include the plural forms as well, unless the context clearly indicates otherwise.

The term “about” means ±10% of the associated numerical value.

### EXAMPLES

### Preparation of Gels

Composition of 4-AAPA azo compound gel is shown in the following Table 4.

| Component | % w/w |
|---|---|
| 4-AAPA diazo compound | 10-50 |
| Hydroxypropylmethyl cellulose | 2 |
| (Hypromellose; Methocel E4M) | |
| EDTA disodium dihydrate, USP | 0.05 |
| Methylparaben, USP/NF | 0.15 |
| QS with purified water | QS to 100 |
| Adjusted by NaOH to pH | |

### Preparation of Foams

Foams are produced by supersaturating a liquid phase with gas. A typical foaming process involves (1) dissolution of the foaming agent, (2) bubble nucleation, (3) bubble growth, and (4) stabilization. Methods to achieve this include whipping, shaking, bubbling, pressurized aerosols and airspray foam pumps.

TABLE 5
Propelling foam APAZA aerosol (INN-108)

| | |
|---|---|
| Mineral oil | 0.5% |
| Isopropyl Myristarte | 0.6% |
| Stearyl alcohol | 1.0% |
| Xanthum gum | 0.3% |
| Methcel K1OOM | 0.3% |
| Tween80 | 1.0% |
| Myrj49 | 3.0% |
| APAZA | 20.0% |
| Methylparaben | 200mg |
| Propylparaben | 50mg |
| Propellant | 8.0% |
| Purified water q.s. | 100% |

1. Aqueous Phase: Dissolve the Xanthan gum, the Methocel K100M, Tween 80, Myrj 49, methyl paraben, propylparaben and APAZA in water, with agitation. The solution is warmed to 50-70° C.
2. Hydrophobic Phase: The isopropyl myristate, stearyl alcohol and mineral oil is added together and warmed.
3. Warm hydrophobic phase is added into the warm aqueous phase with agitation, followed by homogenization.
4. Mixture to cool to room temperature and then add to an aerosol container along with the propellant and is sealed and labeled.

### Alcohol-Free Propelling Foam for Rectal Delivery

Foam concentrate is prepared by mixing different quantities of xanthan gum (0% to 2% w/w) and di-sodium EDTA with water at 55° C. along with 4-AAPA diazo compound. Methyl paraben is dissolved separately in Brij L-23 at the same temperature. The hydrophobic phase is gradually added to the aqueous phase. Propellant A-70 (a hydrocarbon propellant) is delivered at 7% w/w.

### Product Concentrates

Solution aerosols are two-phase systems containing the product concentrate in a propellant, a mixture of propellants, or a mixture of propellant and solvent. Solvents may also be included in the formulation to retard the evaporation of the propellant. Solution aerosols can be difficult to formulate because many propellant or propellant-solvent mixtures are nonpolar and are poor solvents for the product concentrate. In addition, there is a limited number of suitable solvents. Ethyl alcohol is a suitable solvent, as well as propylene glycol, dipropylene glycol, ethyl acetate, hexylene glycol, and acetone.

Aerosol solutions may contain from about 50% to about 90% propellant for topical aerosols and up to 99.5% propellant for oral and nasal aerosols. As the percentage of propellant increases, so does the degree of dispersion and the fineness of the spray. As the percentage of propellant decreases, the wetness of the spray will increase. The particle sizes of the sprays can vary from 5 to 10 μm in inhalation aerosols and 50 to 100 m for topical sprays.

Suspensions aerosols can be made when the product concentrate is insoluble in the propellant or mixture of propellant and solvent, or when a co-solvent is not desirable. Anti-asthmatic drugs, steroids, and antibiotics are delivered as suspension aerosols. When the valve is actuated, the suspension formulation is emitted as an aerosol and the propellant rapidly vaporizes and leaves a fine dispersion of the product concentrate.

Formulation considerations for suspension aerosols, not necessary with solution aerosols, include agglomeration, particle size growth, valve clogging, moisture content, and particle size of the dispersed aerosolized particles. Lubricants such as isopropyl myristate and light mineral oil, and surfactants such as sorbitan trioleate, oleic acid, and lecithin can be used to overcome the difficulties of particle size agglomeration and growth, which are directly related to the clogging problems. The moisture content of the entire formulation should be kept below about 200 to about 300 ppm so all of the ingredients need to be the anhydrous form of the chemical or be capable of becoming anhydrous after a drying process. The particle size of the insoluble product concentrate ingredients should be targeted in the 40 to 50 μm range.

The product concentrate in an emulsion aerosol will comprise the active ingredient(s), aqueous and/or nonaqueous vehicles, and a surfactant. Depending on the components, the emitted product can be a stable foam (shaving cream type) or a quick breaking foam. A quick breaking foam creates a foam when emitted from the container but the foam collapses in a relatively short time. This type of foam can be used to apply the product concentrate to a large area without having to manually rub or spread the product. Also, the active drug is more rapidly available because the foam quickly collapses.

Foams are produced when the product concentrate is dispersed throughout the propellant and the propellant is in the internal phase; i.e., the emulsion behaves like o/w emulsions. When the propellant is in the external phase (i.e., like a w/o emulsion), foams are not created but sprays or wet streams result. Stable foams are produced when surfactants are used that have limited solubility in both the organic and aqueous phases. Surfactants concentrate at the interface between the propellant and the aqueous phase forming a thin film referred to as the “lamella.” It is the specific composition of this lamella that dictates the structural strength and general characteristics of the foam. Thick and tightly layered lamellae produce very structured foams that are capable of supporting their own weight.

Surfactants used in emulsion aerosols can include fatty acids saponified with triethanolamine, anionic surfactants, as well as nonionic surfactants such as the polyoxyethylene fatty esters, polyoxyethylene sorbitan esters, alkyl phenoxy ethanols, and alkanolamides. The nonionic surfactants present fewer compatibility problems because they have no electronic charge.

### Aerosols

Aerosols can be used to administer drugs topically and into body cavities such the vagina and rectum. Pulmonary, nasal, and oral administration of aerosols is intended to achieve either local or systemic therapeutic effect, while topical, vaginal, and rectal administration is only intended for local effect.

Topical aerosol formulations are available for local anesthetics, antiseptics, germicides, first aid preparations, and spray on protective films. These aerosols deliver particles that are larger and more course than the inhalation aerosols. Topical aerosols deliver the active drug in the form of a powder, a wet spray, a stream of liquid, or an ointment like product.

A rectal foam product may be provided with an application device, which is filled with foam. When a valve is actuated and the device is placed in the rectum, the foam is instilled with the device plunger.

1. A method of treating or preventing inflammation of the lower gastrointestinal tract in a subject, comprising rectally administering an effective amount of a pharmaceutical composition comprising an agent that forms one or more 4-AAPA compounds or 5-ASA compounds by azo reduction.

2. The method of claim 1, wherein the subject has colitis.

3. The method of claim 1, wherein the subject has ulcerative colitis or Crohn's disease.

4. The method of claim 1, wherein the subject has or is at risk of pouchitis.

5. The method of any one of claims 1 to 4, wherein the agent is a compound of the formula: 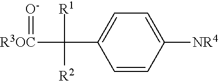
where R¹, R², and R³ are independently hydrogen, halogen, or C1 to C4 alkyl, and R⁴ is: 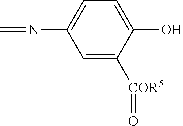
where R⁵ is selected from hydrogen, halogen, and C1 to C4 alkyl; or R⁴ is: 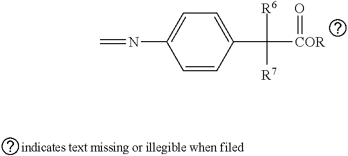
where R⁶, R⁷, and R⁸ are independently hydrogen, halogen, or C1 to C4 alkyl.

6. The method of claim 5, wherein the compound is a 4-aminophenylacetic acid azo bonded dimer.

7. The method of claim 5, wherein the compound is 5-(4-carboxymethyl-phenylazo)-2-hydroxy-benzoic acid.

8. The method of claim 5, wherein the composition comprises a mixture of 5-(4-carboxymethyl-phenylazo)-2-hydroxy-benzoic acid and 4-acetylAPAA or 4-APAA

9. The method of any one of claims 1 to 8, wherein the pharmaceutical composition comprises:
at least one stabilizer or surfactant,
at least one thickener or foaming agent, and
at least one suspending or a solubilizing agent.

10. The method of claim 9, wherein:
at least one of the stabilizer or surfactant is selected from cetyl alcohol, coconut oil, stearic acid, hydroxypropyl methylcellulose, steric acid, citric acid, disodium ethylenediaminetetraacetic acid, and castor oil; and/or
at least one of the foaming agent or thickener is selected from poloxamer F68, polyoxyl stearyl ether, carbomer, polyethylene glycol, caprylic glycerides, cellulose, xanthan gum, and agar; and/or
at least one suspending or solubilizing agent is selected from propylene glycol, glycerine, emulsifying wax, and a solvent optionally selected from one or more of water and alcohol.

11. The method of claim 9 or 10, wherein
the at least one stabilizer or surfactant is about 0.01 to about 0.1%, and optionally about 0.05%, by weight,
the at least one thickener or foaming agent is about 0.05% to about 4% by weight, and
the at least one suspending or solubilizing agent is about 0% to about 30% by weight.

12. The method of any one of claims 1 to 11, wherein the pharmaceutical composition is a foam or foamable composition.

13. The method of claim 12, wherein the foam or foamable composition comprises a propellant.

14. The method of any one of claims 1 to 11, wherein the pharmaceutical composition is a gel or emulsion.

15. The method of any one of claims 1 to 11, wherein the pharmaceutical composition is a suppository.

16. The method of any one of claims 1 to 11, wherein the pharmaceutical composition is a rectal suspension.

17. The method of any one of claims 1 to 16, wherein the subject is suffering from acute pouchitis.

18. The method of any one of claims 1 to 16, wherein the subject is suffering from chronic pouchitis.

19. The method of claim 17 or 18, wherein the composition is administered during a cycle of antibiotic treatment, or between cycles of antibiotic treatment.

20. The method of claim 17 or 18, wherein the composition is administered as an alternative to antibiotic therapy.

21. The method of any one of claims 17 to 20, wherein the composition is administered with probiotic therapy.

22. The method of any one of claims 1 to 21, wherein the subject has pouchitis that is in remission.

23. The method of claim 22, wherein the composition is administered with probiotic therapy.

24. The method of any one of claims 1 to 16, wherein the subject is suffering from antibiotic refractory pouchitis.

25. The method of claim 24, wherein the composition is administered with probiotic therapy.

26. The method of any one of claims 1 to 16, wherein the pharmaceutical composition is administered after a surgical procedure that forms the ileo-anal pouch, and prior to development of pouchitis.

27. The method of claim 26, wherein the subject further receives probiotic therapy.

28. The method of any one of claims 1 to 27, wherein the composition is administered at least once daily.

29. The method of claim 28, wherein the composition is administered for at least one week.

30. A pharmaceutical composition for rectal administration of a 4-AAPA azo compound comprising:
an effective amount of a 4-AAPA azo compound,
at least one stabilizer or surfactant,
at least one thickening or foaming agent, and
at least one suspending or solubilizing agent.

31. The pharmaceutical composition of claim 30, further comprising a propellant.

32. The pharmaceutical composition of claim 30 or 31, wherein:
at least one of the stabilizer or surfactant is selected from cetyl alcohol, coconut oil, stearic acid, hydroxypropyl methylcellulose, steric acid, citric acid, disodium ethylenediaminetetraacetic acid, and castor oil;
at least one of the foaming agent or thickener is selected from poloxamer F68, polyoxyl stearyl ether, polyethylene glycol (PEG) 6 caprylic glycerides, cellulose, xanthan gum, and agar; and
at least one suspending or solubilizing agent is selected from propylene glycol, glycerine, emulsifying wax, and solvents optionally selected from water and/or alcohol.

33. The pharmaceutical composition of claim 32, wherein:
the at least one stabilizer or surfactant is about 0.01 to about 0.1% by weight, and optionally about 0.05% by weight,
the at least one thickener or foaming agent is about 0.05 to about 4% by weight, and
the at least one suspending or solubilizing agent is about 0 to about 30% by weight.

34. The pharmaceutical composition of claim 33, further comprising a propellant or propelling agent at from about 5% to about 50% by weight

35. The pharmaceutical composition of any one of claims 30 to 34, wherein the pharmaceutical composition is a foam or foamable composition.

36. The pharmaceutical composition of claim 35, wherein the pharmaceutical composition is a propelling foam or foamable composition dispersed with a propellant.

37. The pharmaceutical composition of any one of claims 30 to 33, wherein the pharmaceutical composition is a gel or emulsion.

38. The pharmaceutical composition of claim 37, wherein the pharmaceutical composition is a rectal suspension.

39. The pharmaceutical composition of any one of claims 30 to 33, wherein the pharmaceutical composition is dispersed in a suppository base.
